(19) United States
(12) Patent Application Publication
(10) Pub. No.: US 2010/0280350 A1
(43) Pub. Date: Nov. 4, 2010

(54) Chinese medicine tele-diagnostics and triage system

(76) Inventor: Xinyu Zhang, Honolulu, HI (US)

Correspondence Address: XINYU ZHANG, 780 AMANA STREET SUITE 108, HONOLULU, HI, 96814, US

(21) Appl. No.: 12/387,558

(22) Filed: May 2, 2009

(51) Int. Cl. A61B 5/05; A61B 5/00; G06Q 50/00; G06F 17/30

(52) U.S. Cl. 600/407; 600/300; 705/2; 707/802; 707/E17.044

(57) ABSTRACT

The present invention is tele-diagnostics and patient triage method and system for the practices of traditional Chinese medicine (TCM) that acquire patient health condition info through 4 diagnostics process, provide pathogenesis analysis (disease cause, progression, projection), and treatment and prescription options remotely. This method and system established medical business modules with standard syntax through the implementation of medical decision support tools such as relational database, lookup tables, calculators, decision trees, manifestation reference charts, case comparison and statistics, and visualization in disease, pattern and syndrome identification, differentiation and determination. It essentially transform complex Chinese medicine practices into a networked, expert supported system with a knowledge base that deliver the needed information to assist a TCM physician to make medical decisions. Used in medical call centers and large general hospitals, this method and system help a plural of physicians quickly screen and diagnose a large number of patients and triage patients to different level of health care resources according to disease, condition and care needed for the patient. This system is to provide the method for TCM practitioner to treat patients of common disease with low risks that does not warrant a face to face diagnostics. This system also integrates with patient location based information to establish a quick referral system that connects call centers with clinics and hospitals nearby to provide face to face physician service and maintain continuity of care. A network of this system can alleviate the increasing pressure of resource shortage in rural and remote areas, particularly in developing countries.

### STATEMENT REGARDING FED SPONSORED R&D

This invention is not sponsored by Federal Research & Development grants or funds.

This invention relates generally to methods, computing programs and apparatus for establishing a Traditional Chinese Medicine (TCM) tele-diagnostics and triage system that can assist a TCM physician to acquire, classify and analyze vital disease parameters from a patient in a remote location. Based upon the symptoms, disease type and severity level, the TCM physician can either provide treatment and prescription to the patient, or refer the patients to home care with care advises, a face to face consultation if necessary or emergency room visits. This invention can bring conventional TCM practices to call center services, help to establish Chinese medical protocols and guidelines, improve the quality and access of patients in rural, remote and special circumstances, and provide a method to integrate TCM healthcare resources to modern communication network and optimize clinical throughput. The intended use of this invention is TCM medical call centers, hospitals and clinics.

Generally, current TCM practices throughout the world are primarily based upon the experiences and knowledge of the individual physician. Software program and information system in TCM for physician practices, medical protocols, decision support, patient medical record, and treatment outcome evaluations are scarce and do not exist in some areas. Lack of these common tools that western medicine has been using for several decades has led to variance in care and quality measurement, increased emphasis on individual TCM practitioner's undocumented experiences, and lower confidence of TCM clinical outcomes.

Another growing issue of current TCM practice and health care in large is the severe shortage of health care resources. In rural, remote areas and after-hour-care (AHC) in urban areas, a large part of the population does not have access to any timely care including basic consultation and simple medication instruction, particularly in India, China and a few other most populous countries. On the other hand, available resources are very concentrated in big metropolitan areas and large hospitals.

Only in recent years, unique capabilities of traditional medicine such as TCM have been recognized in chronic, preventive and personalized care. Integrated medicine becomes the forefront of research and study. From late 1950's, China has started to integrate western medicine with TCM in clinical practices. With additional computation tools, their experiences and outcomes can be efficiently shared with their peers.

Further, although health care call centers in developed countries have increasingly adopted call center medical triage protocols and software developed by Schmitt and Thompson Clinical Centers and others, medical call centers in China are largely manned by health care practitioner without TCM diagnostics and triage tools.

In summary, current TCM practices for telemedicine and call centers are plagued with inefficiencies and require technological innovations to improve its quality, access, and efficiency.

### CROSS REFERENCES TO RELATED APPLICATIONS

This application claims an invention which was disclosed in Provisional Application No. 61/126,780, filed on May 5, 2008, entitled “Chinese Medicine Tele-Diagnostics and Triage System”. The benefit under 35 USC §119(e) of the United States provisional application is hereby claimed, and the aforementioned application is hereby incorporated herein by reference.

The present invention addresses the aforementioned problems by providing a TCM tele-diagnostic and triage method and system (TCM-TDT) that facilitate the TCM remote diagnostic and treatment decision process. This method includes: (1) medical devices and sensors that acquire necessary health information such as vital signs, imaging streams, acoustic and odors, (2) computer software, standalone or networked, that can collect and record patient medical information, process them with medical business logics and algorithm, provide physician disease/syndrome/severity information based upon proven TCM medical protocols; (3) database structure that contains standard TCM disease coding, reference cases and medical protocols; (4) necessary interface to nature, environmental and public health info that are in the proximity of the patient.

One aspect of the invention is the development of a medical decision support system for TCM. Despite its proven record of effectiveness through history and current practices in China and most part of Asia, TCM is recognized as merely “complementary” medicine to western medicine in North America and Europe in recent years largely because of its unique thinking model and huge cultural, language differences. New advances in “Integrated Medicine” require putting the use of TCM in the framework of evidence based medicine practices. A network based TCM decision support system that allow Extract, Transform, and Load (ETL) of existing TCM cases to a database and incorporate new cases will help to build the necessary knowledge base, repository and data warehouse that is capable of moving into this direction.

Another aspect of the invention is the development of database structure, business logics and algorithm around TCM practices.

Another aspect of the invention is transforming TCM clinician practices into standard medical clinical contents and protocols that determine the disease type, syndrome, and severity level, and thus provide TCM medical disposition.

Another aspect of the invention is treating patient of low risk or sub-health state directly and providing diagnostics, laboratory, prescription and care advices from a call center as a replacement for PCP face to face consultation.

Another aspect of the invention is the use of TCM decision support system and medical protocols in call center operations.

Another aspect of the invention is the use of a internet based networked system that allows a TCM physician with less expertise in rural or remote clinics to tap into cases, experiences, and judgments of other TCM experts in metropolitan areas. They can also take advantages of the collective intelligence of a TCM peer group.

Further aspects of this invention will become apparent in the Detailed Description and by reference to the attached drawings.

### DESCRIPTION OF THE FIGURES OF THE DRAWINGS

These diagrams describe scenarios for airport operators although the methods, algorithms and computer programs can be applied into many other scenarios.

FIG. 1 is a block diagram of the essential components that a TCM physician can interact with a patient remotely through audio/video/data collection equipments, sensors and communication media.

FIG. 2 illustrates the processes, methods and software modules of the TCM tele-diagnostics and triage system in physician's remote office.

FIG. 3 shows that a TCM decision support system takes data inputs from a plural of diagnostics methods and produce the disease and syndrome outputs.

FIG. 4 shows that a TCM physician uses the Observation/Inspection diagnostic method to examine the conditions of a patient's overall body.

FIG. 5 shows that a TCM physician uses the Observation/Inspection diagnostic method to examine the conditions of a patient's specific body parts.

FIG. 6 shows that a TCM physician uses the Auscultation/Olfaction diagnostic method to determine a patient's disease and syndrome.

FIG. 7 shows that a TCM physician uses the Pulse-taking and Palpation diagnostic method to examine the conditions of a patient's internal organs.

FIG. 8 shows that a TCM physician uses the Inquiry diagnostic method to determine a patient's medical conditions.

FIG. 9 illustrates a decision tree that a TCM physician uses in Inquiry diagnostic method to determine a patient's disease and syndrome.

FIG. 10 shows the processes and algorithms that TCM physician uses to determine disease family and disease type of a patient.

FIG. 11 shows the methods, process and logics that TCM physician determines the patient's pattern, syndrome and severity/stage of the disease.

FIG. 12 illustrates treatment logics, methods, options, associated prescription and formulas.

### FEATURE DESCRIPTION

In the following description, reference is made to the accompanying drawings, which form a part hereof, and which show, by way of illustration, specific embodiments or processes in which the invention may be practiced. Where possible, the same reference numbers are used throughout the drawings to refer to the same or like components. In some instances, numerous specific details are set forth in order to provide a thorough understanding of the present invention. The present invention, however, may be practiced without the specific details or with certain alternative equivalent devices and methods to those described herein. In other instances, well-known methods and devices have not been described in detail so as not to unnecessarily obscure aspects of the present invention.

### I. Patient Complaint and TCM Diagnostics

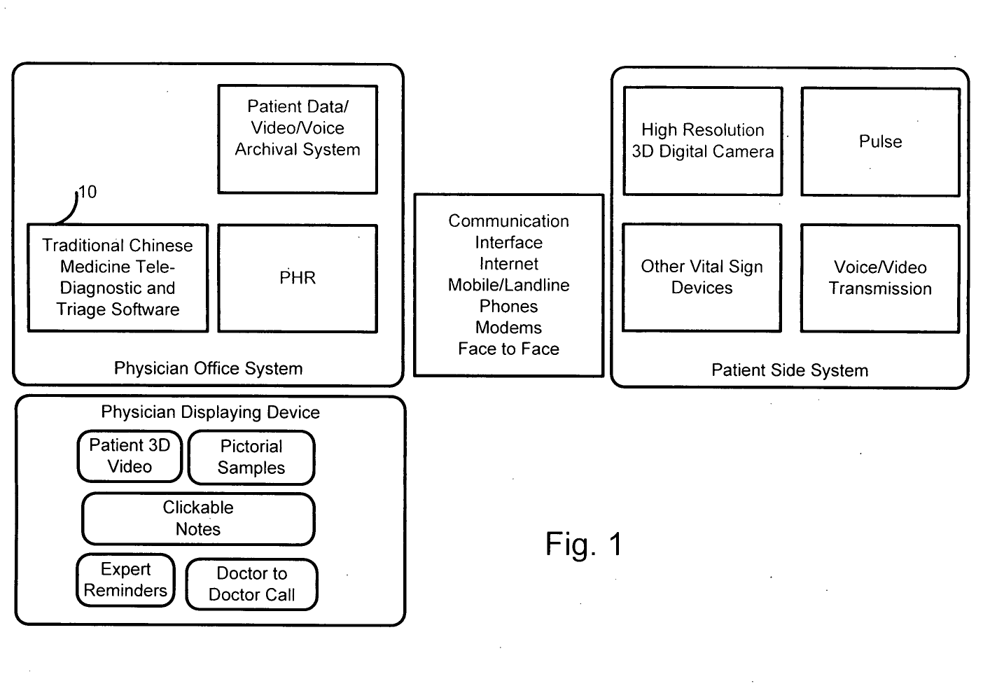

FIG. 1 illustrates a telemedicine system that is commonly used in applications such as Cisco Health Presence, American Well online care system or medical home concepts. This invention integrates TCM with a similar system with a TCM Tele-diagnostic and Triage Software (10).

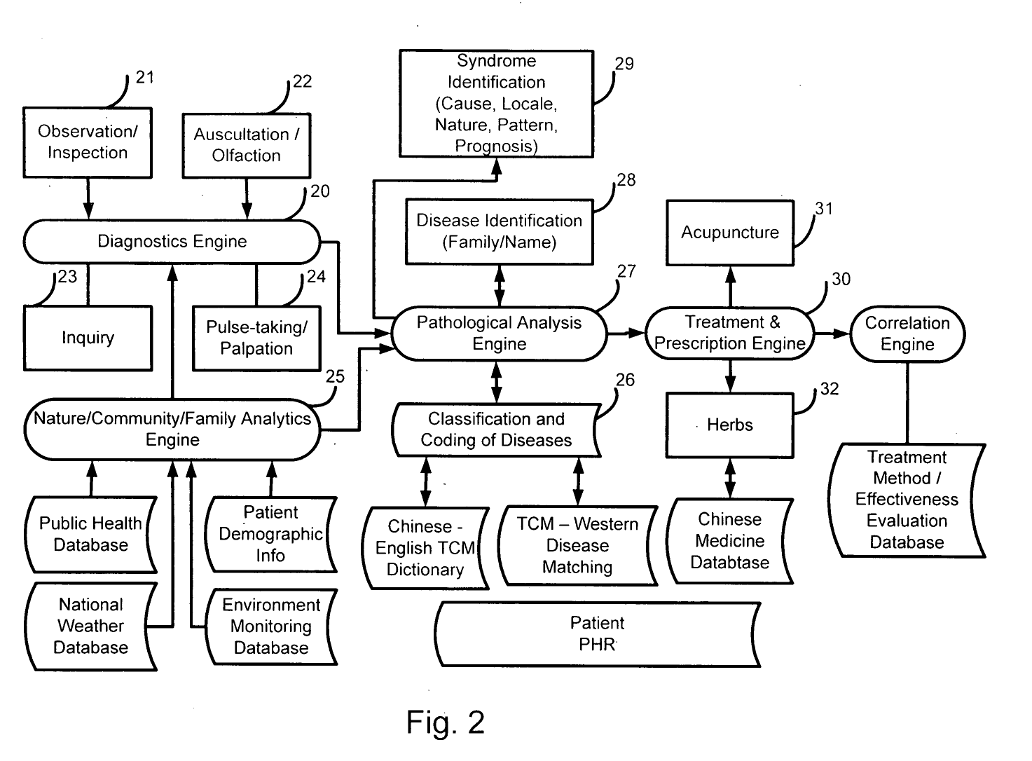

In FIG. 2, the diagnostic engine (20) has four diagnostic methods: Inspection (i.e. [symbol] in simplified Chinese) (21), Auscultation and Olfaction (i.e. [symbol] in simplified Chinese) (22), Inquiry ( [symbol] i.e. in simplified Chinese) (23), Pulse-taking and Palpation (i.e. [symbol] in simplified Chinese) (24). They are the four basic procedures used in diagnosing a disease by a TCM physician. They examine pathological conditions of a patient from different angles and aspects and find out their etiology and pathogenesis. Each procedure has its specific function that cannot be substituted for the others and information gathered from four methods is correlated and supplemental to each other.

One medical study states that the reliability of diagnostics through each of these four methods if used separately is 85%, 80% to 100%, 70% and 90% respectively. In clinical practice, only when the four procedures are organically integrated, a disease can be fully examined and thus, a correct diagnosis can be made.

Inspection (21) is the diagnostic procedure that a TCM physician may observe the patient's vitality, complexion, physical build, head, neck, five sense organs, skin, tongue, external genitalia and anus on purpose so as to understand the condition of a disease. A high quality image and video stream acquiring devices can be used to extract patient info, transmitted to the physician office through interne or other communication media, stored in the patient electronic medical records. TCM-TDT can use video/image object identification and comparison technology to search for similar physical properties, signs and symptoms of these attributes. The physician can choose the most appropriate one based upon recommendation and options of the system.

Auscultation (22) is the procedure that a TCM physician listens to the patient's voice, speaking, respiration, coughing and moaning to understand the changes of the phonology organ and to infer the pathological development of the internal organs. This can be achieved through acoustic sensors or microphones of high quality. Olfaction (22) means smelling the patient's odor such as breathing and discharge. Capability of sensors of this field is getting better and yet can not be compared with that of an experienced TCM practitioner. Additional assistance of a healthcare worker such as a nurse may be needed in the remote office with the patient.

Inquiry (23) is a diagnostic method that a physician inquires the patient or caretakers to collect the information concerning a disease. This procedure in TCM is not much different from a western medicine PCP inquiry procedure. However, over thousands of years of practices, TCM physician-patient questions are more in volume and more systematical than western medicine. The scope of inquiry includes the chief complaints, present case, past medical history, and other relevant disease information. TCM has proven methods such as “10 Inquiry”, which is and/or can be developed into medical protocols with systematical decision trees and lookup tables. The inquiry can be conducted easily through any telecommunication media in call center.

Pulse-taking and Palpation (24) is the diagnostic method including pulse-taking and in some cases palpation of different parts of the body. Capability, of sensors of this field is getting better and yet can not be compared with that of an experienced TCM practitioner. Additional assistance of a healthcare worker such as a nurse may be needed in the remote office with the patient.

TCM was one of the few traditional medical practices that take nature and environmental factors into diagnostics. Nature/Community/Family Analytical Engine (25) feeds data into Diagnostics Engine (20). These data are analyzed by pathological analysis engine (27) to determine disease identification (28) and syndrome identification (29). These engines can reference databases in TCM classification and coding of diseases (26).

Disease, syndrome, and severity level are bases for TCM physicians to use treatment and prescription engine (30) to decide which of the several major treatment categories (i.e. Acupuncture (31) or Herbs (32)) will be best fit for patient conditions.

This invention created business logics, algorithms and methods that assist a physician to correlate and have a complete and comparative understanding of the disease symptoms, signs, causes and syndrome information that are collect and recorded. These tools include EMR, questionnaire similar to SF-36, relational database structure, organ manifestation charts, differentiation lookup tables, and decision trees and visualization tools to present actionable info to the physician.

### II. Disease and Syndrome Determination

TCM diseases are classified into 7 families ( [symbol] in simplified Chinese) in Classification and Codes of Diseases and Zheng Of Traditional Chinese Medicine, the China National Standard GB/T15657-1995. Each family contains a plural of disease specialty types [symbol] [symbol] in simplified Chinese) that are associated with type of patients, body parts and organs, conditions of the disease. They are not directly equivalent to their western medicine counterparts. Under each type of disease type, disease is further identified by a plural of disease names ( [symbol] in simplified Chinese), some of which are equivalent or very close to their western disease counterparts. TCM-TDT incorporates this standard and creates table, fields and records in a relational database with index to be used in the diagnostics and treatment process.

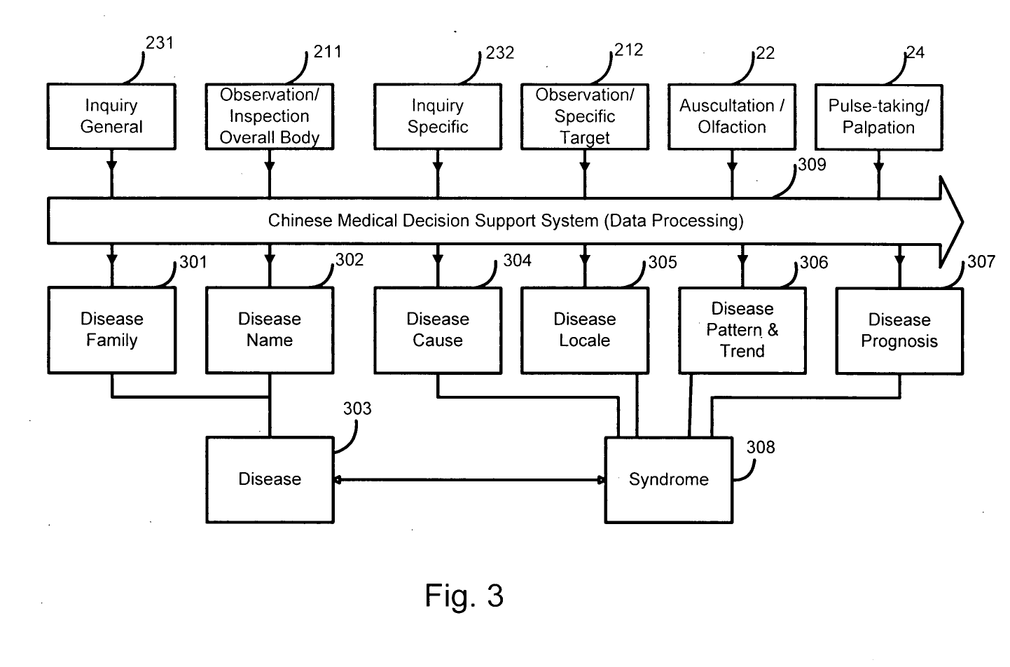

FIG. 3 illustrates a general process that a TCM PCP would take in a physician-patient encounter and the software modules in the system that will be needed. Inquiry General (231) and Overall Body Observation (211) diagnostics procedures are to identify disease family (301) and disease name (302). The other procedures (232, 212, 22, and 24) are to determine syndrome—disease cause (304), locale (305), pattern & trend (306) and prognosis (307). A syndrome (308) describes a life cycle of the disease and its impact on the patient.

The data processing engine (309) utilizes decisions trees, lookup tables and organ manifestation reference charts to quickly triage patients into family and type of disease. Then the physician uses Bayesian or statistical case analysis in other diagnostic procedures to determine the syndrome.

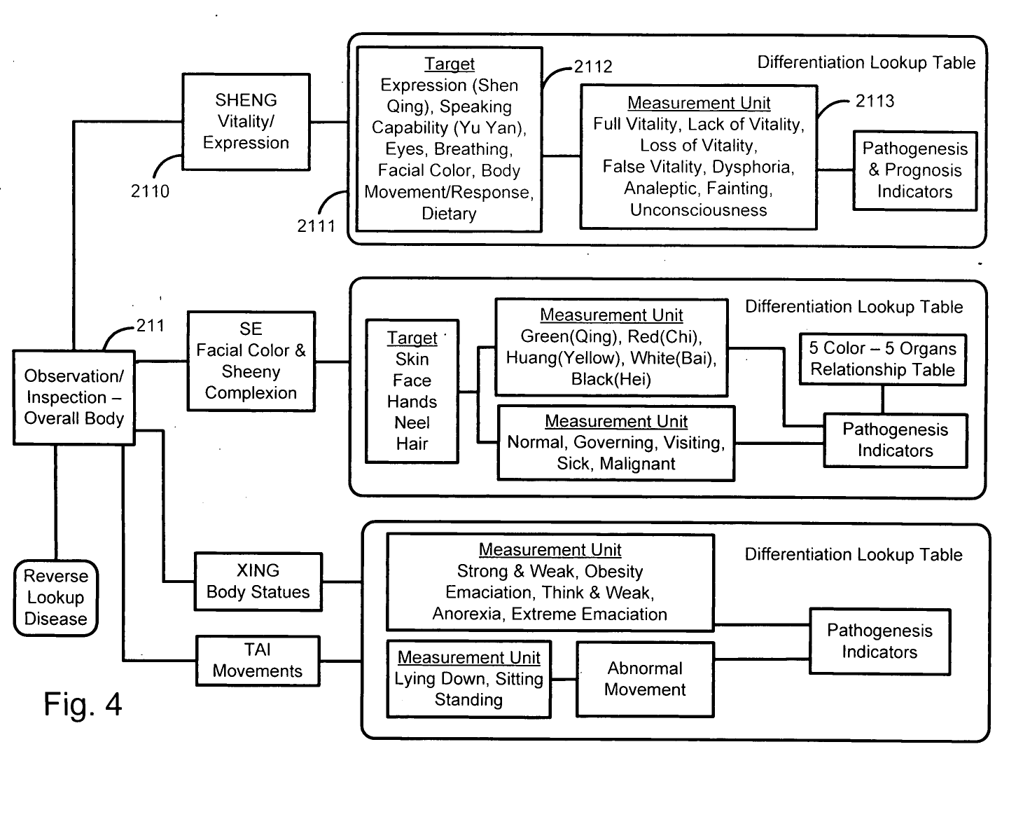

FIG. 4 illustrates how the observation/inspection procedure (211) uses differentiation lookup table (2111) as a diagnose tool to collect data for pathological analysis. Procedure observation/inspection-overall body (211) examines four key elements of patient's appearance. Vitality/expression (2110) has a series of target (2112) attributes that needs to be examined. The TCM physician assign each attribute with measurement unit (2113). The target and measurement unit will provide data to determine the disease, syndrome and severity level.

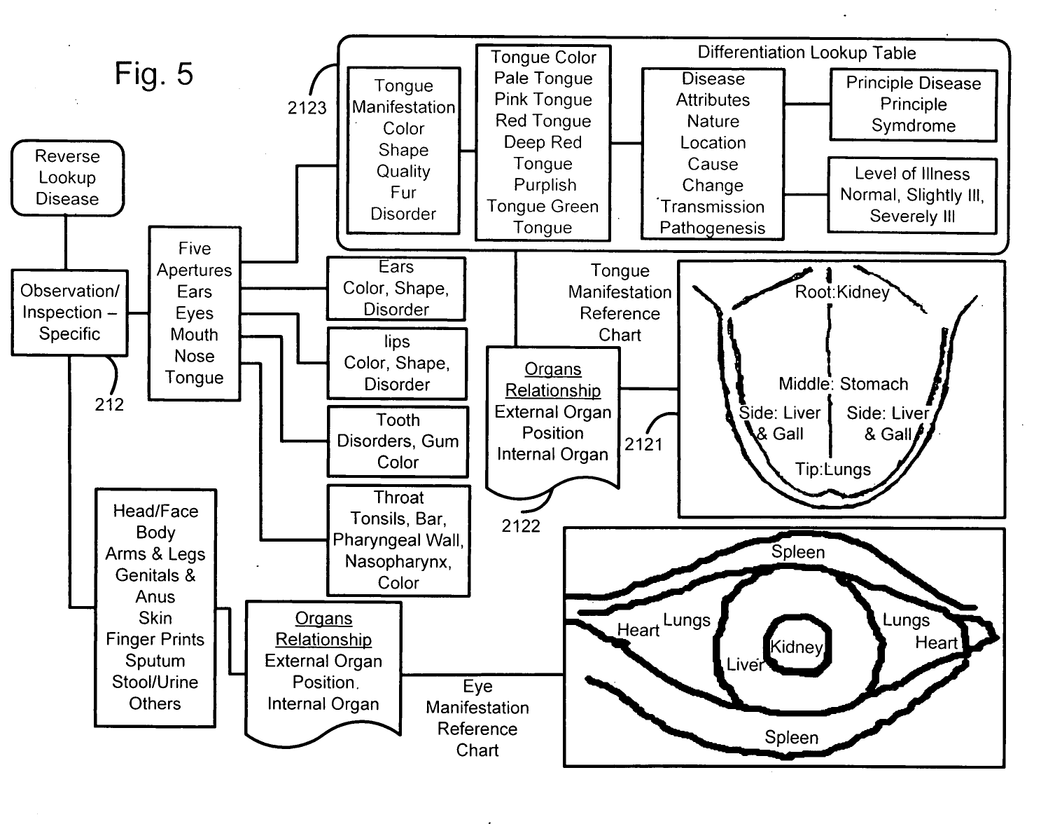

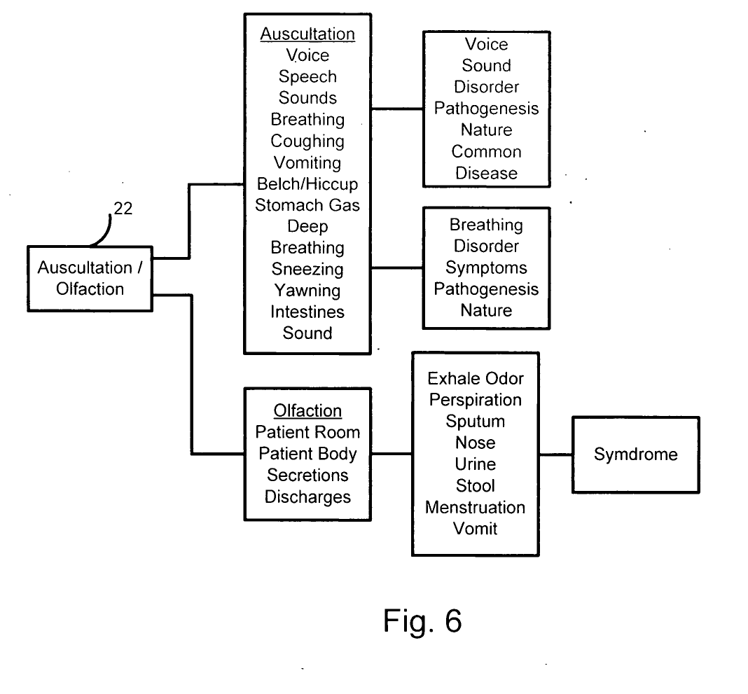

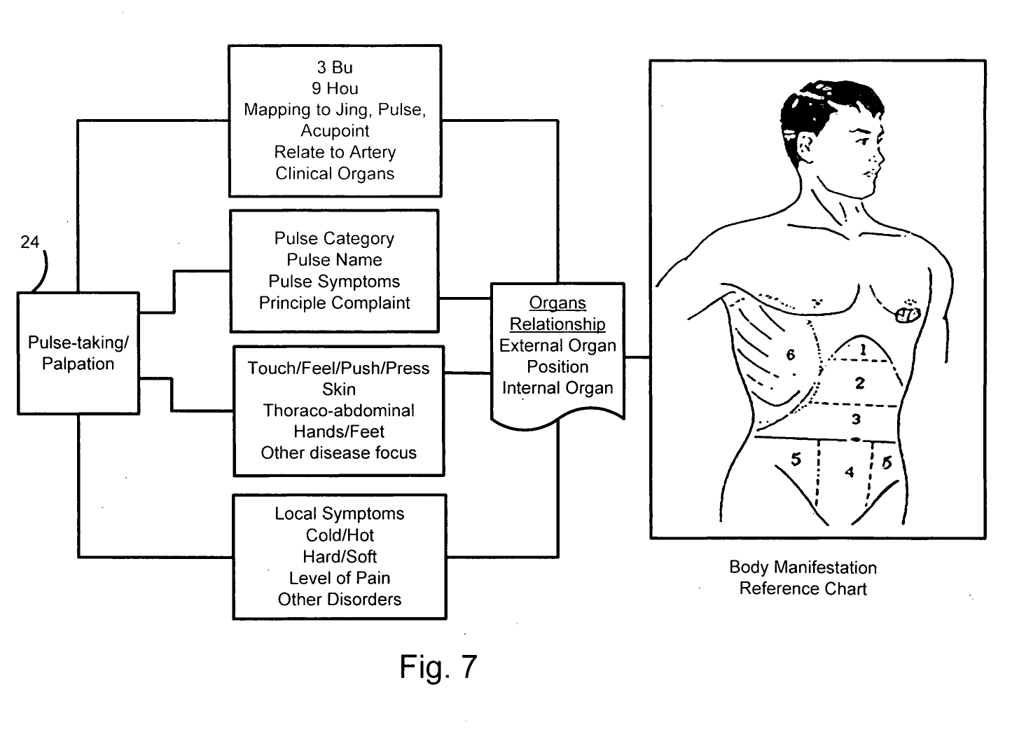

FIG. 5 illustrates another diagnostic tool—manifestation reference chart (2121). A manifestation reference chart (2121) is a pictorial reference chart that TCM physician uses to examine physical properties of an aperture such as tongue to analyze conditions of internal organs. Different locale and parts of the tongue in chart (2121) is linked to different internal organ in the organs relationship (2122). This assists the physician to quickly drill down severity levels in differentiation lookup table. FIG. 6 and FIG. 7 uses similar diagnostic tools for the auscultation/olfaction (22) and pulse-taking/palpation (24) procedures.

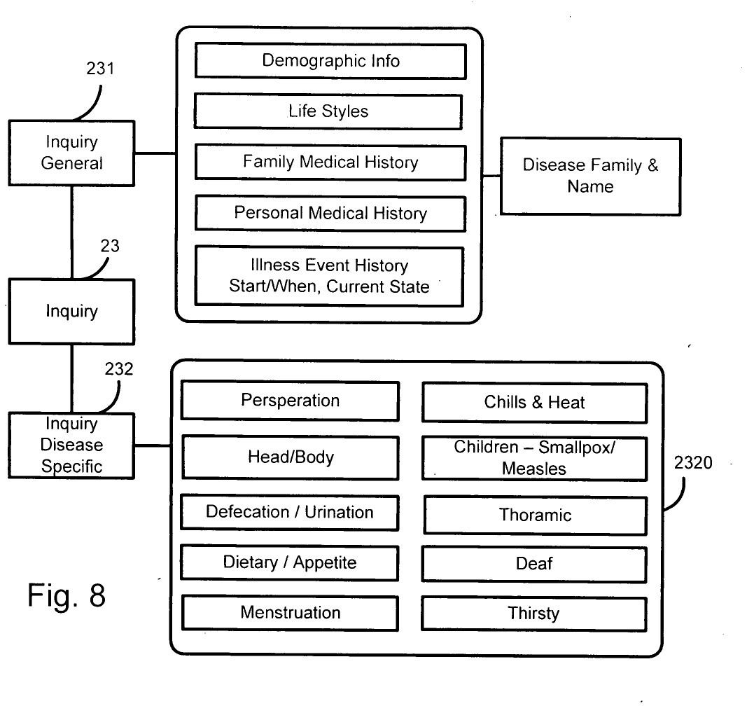

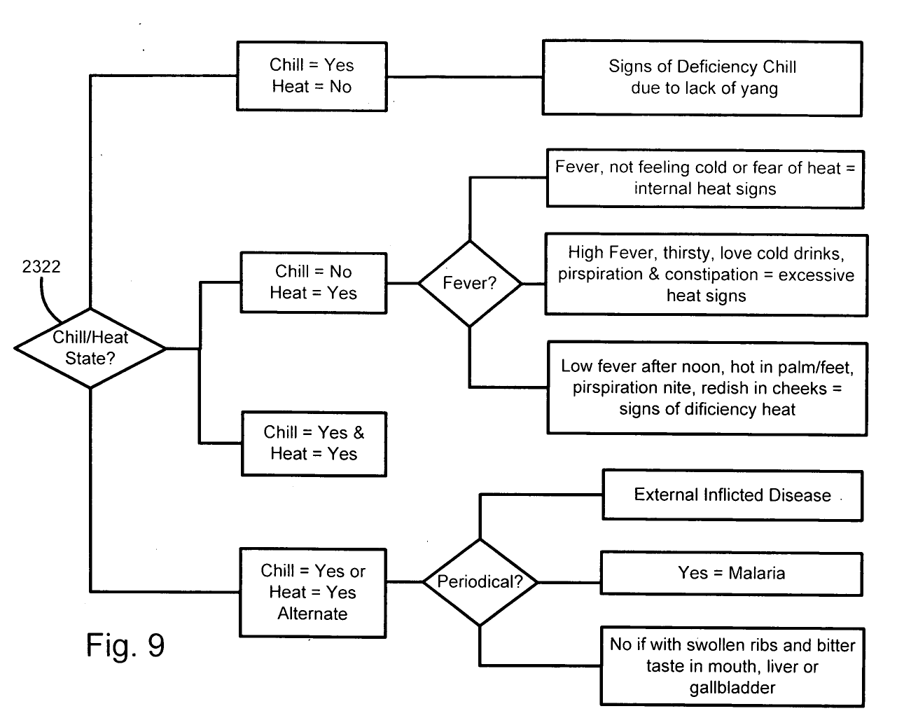

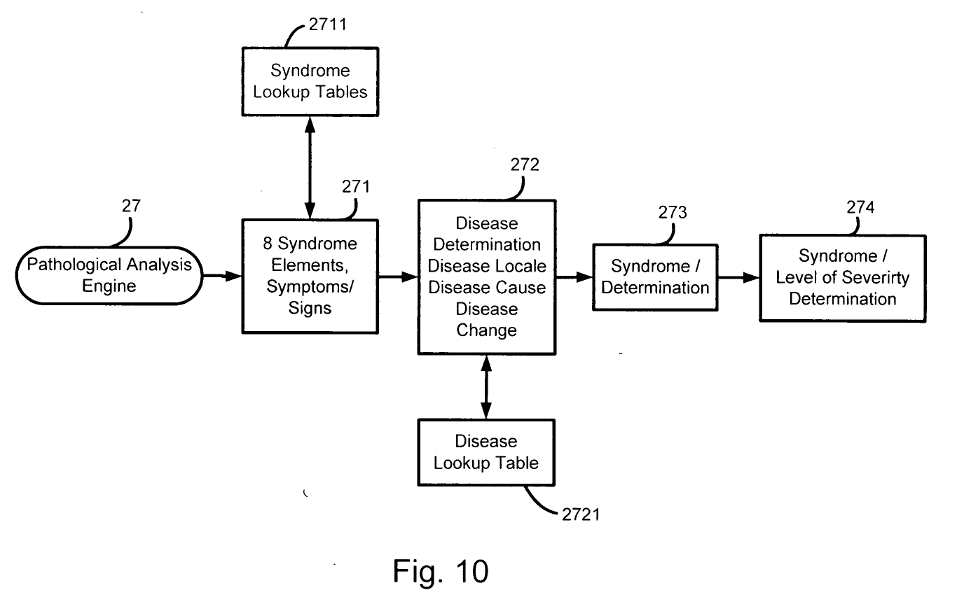

FIG. 8 and FIG. 9 illustrate the inquiry procedure (23). The inquiry general procedure (231) is used to determine disease family and disease name. The inquiry disease specific procedure (232) is to determine syndrome and severity level. The “10 inquiry” (2320) has 10 categories of questions and the procedure is proven to be effective. For example, one of category is to probe the patient with questions around the syndrome element of chills/heat state. Based upon the responses of the patient, a decision tree (2322) can be drawn to determine the disease, syndrome and severity level. The data collected in this process can be recorded in a syndrome lookup table (2711) as illustrated in FIG. 10.

The most critical process of diagnosing a patient in TCM is to determine the disease name, the syndrome and severity of the disease. FIG. 10 expands on how to use data collected via diagnostics engine to determine syndrome. In this process, TCM physician can first use (syndrome elements, symptom/sign) data pairs (271) in the syndrome lookup table (2711) to conduct a pathological analysis. This information and other data collected in the 4 diagnostics procedures will linked to a specific disease family and name (272) in disease lookup table (2721). Syndrome lookup table (2711) as illustrated in Table 1 and disease lookup table (2721) as illustrated in Table 2 can further correlate to disease locale (i.e. internal, external, or location on the organ), the disease cause and disease condition changes.

TABLE 1
Syndrome Lookup Table

| Syndrome | | | | | | | | | | |
|---|---|---|---|---|---|---|---|---|---|---|
| Element | Symptoms/Signs | | | | | | | | Cause | Change |
| | Chill/ | Other | Urine/ | Tongue | Pulse | | | | | |
| | Heat | Symptoms | Stool | | | | | | | |
| Exterior/ | | | | | | | | | | |
| Inner | | | | | | | | | | |
| | Chill/ | Mouth | Facial | Vitality/ | Sputum/ | Urine/ | Tongue | Pulse | | |
| | Heat | Thirsty | Color | Posture | Mucus | Stool | | | | |
| Chill/ | | | | | | | | | | |
| Heat | | | | | | | | | | |
| | Chill/ | Mouth | Facial | Perspi- | Vitality/ | Thoracic | Urine/ | | | |
| | Heat | Thirsty | Color | ration | Posture | Abdominal | Stool | | | |
| Deficiency/ | | | | | | | | | | |
| Excessive | | | | | | | | | | |
| | Perspi- | Arms & | Other | Tongue | Pulse | | | | | |
| | ration | Legs | Symptoms | | | | | | | |
| Ying/Yang | | | | | | | | | | |

TABLE 2
Disease Lookup Table

| Family | Disease Name | | | | | | | |
|---|---|---|---|---|---|---|---|---|
| Internal | Pulmonary | Cardiology | Spleen | Hepatic | Nephrology | Exogenous | Parasite | Oncology |
| Medicine | | | | | | Heat | | |
| External | Ulcer And | Breast | Genitalia | Dermatology | Anus & | Oncology | Cancer | Others |
| Medicine | Sore | | | | Intestines | | | |
| Gynecology & | Myopathy | Leucorrhoea | Pregnancy | Puerperal | Oncology | Cancer | Others | |
| Obstetrics | | | | | | | | |
| Pediatrics | Neonatal | Seasonal | Misc | Parasites | Oncology | Cancer | | |
| Ophthalmology | Eyelid | Canthus | Bulbar | Cornea | Pupil | Surgical | Oncology | Cancer |
| | | | Conjunctiva | | | | | Others |
| Otorhinolaryng/ | Ear | Nasal | Throat | Mouth & Tooth | Oncology | Cancer | | |
| Stomatology | | | | | | | | |
| Orthopedics & | Fracture | Dislocation | Tissue | Internal | Injuries | Oncology | | |
| Traumatology | | | | Disorder Due | | | | |
| | | | | To Injury | | | | |

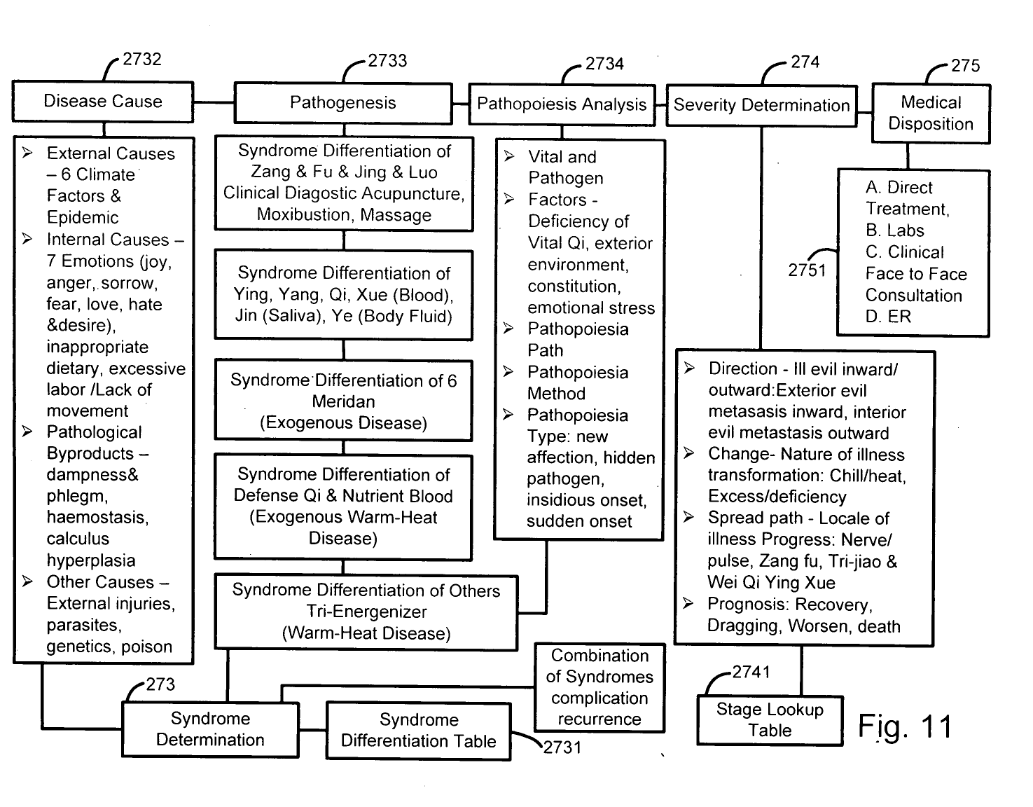

FIG. 11 illustrates how TCM physician uses syndrome determination to further define disease nature and severity level. The concept of syndrome (273) is unique to TCM. GB/T15657-1995 standard classifies all diseases into 6 pattern/syndrome families ( [symbol] in simplified Chinese) (2731), namely disease cause, ying-yang-qi-blood-fluid-humor, zang-fu-jing-luo, 6 meridians, defense-qi-nutrient-blood, and others based upon the traditional 6 pattern and syndrome identification and differentiation methods. Each syndrome family has different attributes ( [symbol] in simplified Chinese) to describe the disease nature and conditions ( [symbol] [symbol] in simplified Chinese). A syndrome name is combination of a primary and secondary attributes as illustrated in Table 3 syndrome differentiation table. TCM-TDT adopts this system with tables, fields, coding methods in a relational database structure. A TCM physician can use this system to conduct disease cause (2732) analysis, pathogenesis (2733) analysis, and pathopoiesis (2734) to what caused the disease and how the disease started.

### III. Disease Severity Determination

As illustrated in FIG. 11, after the physician determines exact disease and syndrome, the next critical step is disease severity determination (274). A TCM physician examines disease conditions in terms of stages of disease development, spreading paths, and the level of severity, criticality, and prognosis.

In TCM-TDT, these tools include stage lookup table (2741) as illustrated in Table 4, decision trees, medical calculators, manifestation reference charts, and statistical case analysis. This step is crucial for call center triage protocols. Depending upon the disease, syndrome and severity level, TCM physician can make medical disposition (275). The physician can directly A) treat common, low risk disease and provide home care advices, or B) request patients to go to labs for testing, or C) refers the patient for face to face consultation, or D) send patient to Emergency Room (ER) (2751).

TABLE 3
Syndrome Differentiation Table

| | | Yin-Yang- | | | Defense, Qi, | |
|---|---|---|---|---|---|---|
| Differentiation | Disease | Qi-Blood- | Zang-Fu- | 6 | Nutrient, | |
| Method | Cause | Fluid-Humor | Jing-Luo | Meridan | Blood | Others |
| Syndrome | Wind | Yin | Heart | Tai Yang | Weifen | Others |
| Attributes | Cold | Yang | Lungs | Yang Ming | Qifen | Stages |
| | Summer Heat | Qi | Spleen | Shao Yang | Yingfen | Shapes |
| | Dampness | Blood (Stasis) | Liver | Tai Yin | Xuefen | |
| | Dryness | Fluid | Gallbladder | Shao Yin | | |
| | Fire | Phlegm | Kidney | Jue Yin | | |
| | Heat | | Small Intestines | | | |
| | Toxin | | Large Intestine | | | |
| | Pathogen | | Stomach | | | |
| | Dietary | | Jing | | | |
| | Emotions | | Luo | | | |
| | External | | Pulse | | | |
| | Injuries | | | | | |

TABLE 4
Disease Stage Lookup Table

| Disease | | | Coughing | Treatment |
|---|---|---|---|---|
| Stage | Sores Stage | Fever Stage | Stage | Stage |
| Early | Ulcer | Becoming | Starting Cough | After |
| | Formation | Feverish | | treatment |
| Halfway | Pustule | Fever | Severe Cough | Prior to |
| | Brewing | Expansion | | treatment |
| Late | Pustule | After Fever | | |
| | Completion | | | |
| | Pustule Burst | Fever Maturing | | |
| | Pustule | | | |
| | Recovery | | | |
| | Pustule After | | | |
| | Burst | | | |

### IV. Treatment & Prescription Options

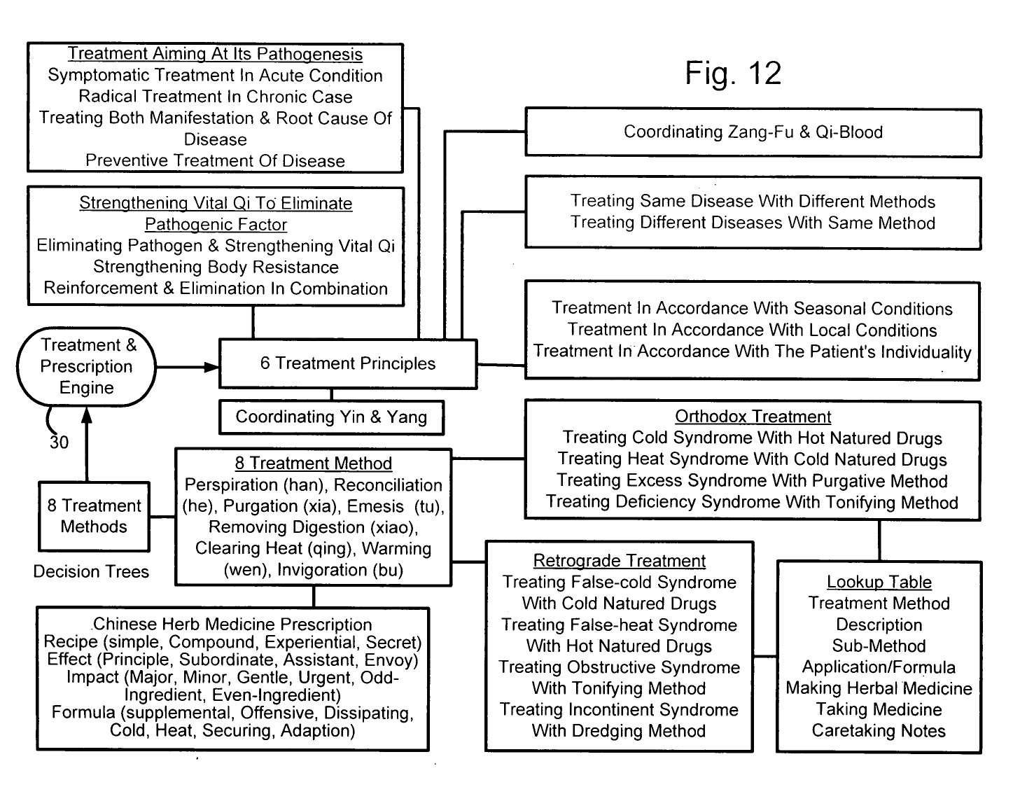

In FIG. 12, corresponding to the disease, syndrome and severity level, for a common, low risk disease directly from remote office (i.e. call center), the TCM physician can use treatment and prescription engine (30). The treatment principles and methods and options are available through lookup tables. Each method is associated with a plural of prescription formulas as illustrated in Table 5. Each formula is associated with a set of properties such as recipes, quantity of each, associated reference pictures for the herbs, how the medicine is prepared, taken with or without meals, and how the patient is cared by care takers. In TCM-TDT, these tools include differentiation lookup tables, decision trees, calculators, and statistical case analysis. Additional tools such as capability of clear instruction will be given in formats such as hard copies and SMS messages that are different from verbal instruction.

TABLE 5
8 Treatment Method Table

| Method | Formula |
|---|---|
| Diaphoresis | Resolving Exterior Method |
| | Promote Eruption |
| | Eliminating Dampness Method |
| | Clearing Heat For Detumescence |
| Emetic Method | Steep Emetic Method |
| | Slope Emetic Method |
| | Exterior Probe Method |
| Purgative Method | Cold Cathartic |
| | Warm Cathartic |
| | Moistened Cathartic |
| | Drastic Diuretics |
| Harmonizing | Reconciling Shaoyang |
| Method | Harmonizing Liver And Spleen |
| | Coordinating Intestines And Stomach |
| | Dispersing Stagnated Liver Qi For Regulating Stomach |
| | Harmonizing Qi And Blood |
| | Harmonizing Yingfen And Weifen |
| Warming Method | Warming Interior For Dispelling Cold |
| | Warming Channel For Dispelling Cold |
| | Emergent Resuscitation |
| Cleaning Heat | Clearing Heat At The Qi Level |
| Method | Clearing Heat-Toxin |
| | Heat-Clearing And Detoxication |
| | Clearing Heat To Nuture Yin |
| | Clearing Heat In The Viscera |

### V. Knowledge Base & Inference Mechanism

TCM-TDT system contains a large knowledge base including TCM standards, clinical medical guidelines, term and definition dictionary, classical documents, and cases.

TCM-TDT uses online TCM experts, known cases, and network based analysis to establish through medical logic modules to build its inference mechanism.

A modified Arden syntax is used in TCM-TDT for TCM.

### VI. Patient Triage in Call Center

Key application of this invention is to triage a plural of patients in call centers for hospitals and clinics. For majority of low risk, common diseases that has established proven medical protocols in the system and face to face consultation is not required, TCM-TDT system provides call center an efficient and effective way that directly treat patients with low severity with prescription. They can also used to provide preventive care advices such as dietary for patients that are in sub-health state and determined not ill. Depending upon the complexity of the problems, this system can integrate with the assistance of a healthcare worker in remote office with remote connectivity module. A networked implementation of TCM-TDT in an administrative domain (region or country) can bring all the healthcare resources into the system, dispatch/allocate these resource based upon disease/severity, and create a physician to physician, physician to patients or patients to patients assistance system. This system can become an organic self-learning system and optimize overall resource usage and throughput.

While certain preferred embodiments have been described and shown in the accompanying drawing, it is to be understood that such embodiments are merely illustrative of, and not restrictive on, the broad invention. Other embodiments that are apparent to those of ordinary skill in the art, including embodiments that do not provide all of the features and advantages set forth herein, are also within the scope of this invention. Further, it is to be understood that this invention is not limited to the specific construction and arrangements shown and described since various modifications or changes may occur to those of ordinary skill in the art without departing from the spirit and scope of the invention. Accordingly, the scope of the invention is defined by the claims that follow. In the claims, a portion shall include greater than none and up to the whole of a thing. In the method claims, reference characters are used for convenience of description only, and do not indicate a particular order for performing the method.

1. A method and a computer software that a Chinese medicine physician can interact with a patient through a plural of medical sensors and a networked computer software program, comprising of:
incorporating weather, environmental, family data as part of medical info;
gathering patient health condition data through 4 diagnostic methods, namely Auscultation and Olfaction, Inquiry, Pulse-taking and Palpation, together with medical sensors;
analyzing cause of disease, progression, and possible outcomes with 8 principles and 6 dialectical methods;
determining the severity and risk of patient and whether it is necessary to have a face to face consultation with a physician;
if not needed, providing treatment options and prescription recommendations;
using Chinese medical body charts and sample cases as references;
a database structure of patient electronic health record established around Chinese medicine with both English and Chinese as primary language;
if needed, determining location data with communication device and linking the system to clinic or hospital for care continuity and pharmacy or labs for special services;
using medical logic modules and tools such as lookup tables, calculators, decision trees, statistics analysis, and visualization tools;
determining subhealth conditions and provide a disease management plan.

2. The Observation and Inspection process and software in claims 1 that the physician can retrieve real time patient image for imaging sensors and gather 4 categories (Shen, Se, Xing, Tai) health condition info, comprising of:
a plural of observation and inspection targets or areas such as whole body, body parts, and their exterior properties including shape, color etc;
a plural of measurement methods and units that determine normal and abnormal and different level of severity;
a link to possible disease cause and nature in different database;
a reference chart and relation established between an internal organs and body parts to external parts so that an ill internal part may be diagnostic;
with references charts and uses cases in database as references to physician;
allowing physician with a few clicks to mark and record comments during the process.

3. The Auscultation and Olfaction process and software module in claims 1 that the physician can collect acoustic and odor data from the patients acoustic and odor sensors, or if necessary through face to face or on site inspection, that comprising of:
a plural of acoustic and odor sensors;
having a list of targets and items such voice and body odor as in a database that the physician will take sample of;
having links to possible disease causes and properties and level of disorders;

4. A Inquiry process in claims 1 that a physician can survey the patients through questions and answers over the phone and other communication media such as web or email, that comprising:
a list of questions that is related to patient demographic info, life styles, family medical history, personal medical history and parameters of this illness event such as the history, starting date and time, and current state;
a list of questions that are related to 10 Inquiry (chills & heat, perspiration, head/body, defecation and urination, dietary/appetite, menstruation, measles, thoracic, deaf and thirsty) Chinese medicine practice method;
a list of clarification and backup questions if the patient could not comprehend initial questions.

5.

6. Pulse-taking and Palpation process that the physician can use sensors or face to face consultation to gather patient information in claims 1, that comprising:
the method of 3 BU 9 HOU that maps pulse and palpation in 3 category 9 key locations provided with a reference chart;
a database structure of pulse category, name symptoms and their links to principle complaints;
palpation method through touch, feel and press in body parts and a list of local symptoms and reference chart that are related to these examination.

7. The 8 principles differentiation methods that a medical logic module to help physician to determines the nature of the illness, that comprising:
a database structure recording the 8 principles (interior/exterior, chill/heat, deficiency/excess, and yin/yang);
a database structure associating the symptoms of different body parts, organs and other property that gathered through 4 diagnostics process;
determining disease family and specialty (type);
disease nature that is described by the 8 principles.

8. The 6 pattern and syndrome identification and differentiation method is a medical logic module and analysis engine that assist the physician to determine cause, orientation, onset, progression, common outcomes, and degree of an illness in claims 1, that comprising of:
cause of illness includes externally inflicted by 6 YIN factors (wind, cold, heat, wet, dry, internal heat) or epidemic disease, internally inflicted by 7 emotions (joy, anger, sorrow, fear, love, hate, desire), pathological byproducts and other causes;
pathogenesis of 6 pattern and syndrome identification and determination (Disease Cause, Ying-Yang-Qi-Blood-Fluid-Humor, Zang-Fu-Jing-Luo, 6-meridian, Defense-Qi-Nutrient-Blood, Others);
onset and processing of becoming ill includes illness factors, path and method and type of pathopoiesis, each pathogenesis corresponds to certain disease areas;
severity of the illness in terms of orientation/direction, change of nature, locale/progression path, level of spreading areas, common outcomes this type of disease and corresponding medical triage options (i.e. (A) treat common, low risk disease and provide home care advices, or B) request patients to go to labs for testing, or C) refers the patient for face to face consultation, or D) send patient to Emergency Room (ER) for a TCM call center operation.

9. Treatment options, methods and prescription formula in claim 1, that comprise of:
6 principles of treatment;
8 methods of treatment including diaphoresis, emetic, purgative, harmonizing, warming, cleaning heat, tonifying and resolving;
prescription method that related formulas constitute of primary, secondary, assistance and execution components;
medical logic modules that include lookup tables, calculators and detailed medication instructions.
